(19) United States
(12) Patent Application Publication
(10) Pub. No.: US 2011/0129586 A1
(43) Pub. Date: Jun. 2, 2011

(54) WHITE WHEAT VARIETIES, AND COMPOSITIONS AND METHODS OF USING THE SAME

(75) Inventors: Charles Ntiamoah, Chandler, AZ (US); Theresa Cogswell, Olathe, KS (US)

(73) Assignees: DOW AGROSCIENCES LLC, Indianapolis, IN (US); WORLD WIDE WHEAT, L.L.C., Tempe, AZ (US)

(21) Appl. No.: 12/953,848

(22) Filed: Nov. 24, 2010

Related U.S. Application Data
(60) Provisional application No. 61/264,599, filed on Nov. 25, 2009.
(60) Provisional application No. 61/405,124, filed on Oct. 20, 2010.
(60) Provisional application No. 61/369,566, filed on Jul. 30, 2010.
(60) Provisional application No. 61/405,071, filed on Oct. 20, 2010.

(51) Int. Cl. A21D 2/00; A01H 5/10; A01H 5/00; C12N 5/04; A01H 1/02; C12N 15/82; A21D 13/00

(52) U.S. Cl. 426/549; 800/320.3; 435/410; 800/260; 800/278; 800/279; 800/300; 800/302; 426/622

(57) ABSTRACT

This invention relates generally to certain improved white wheat varieties, and grain products comprising said improved white wheat varieties. In certain embodiments, wheat varieties are provided that exhibit advantageous traits of white coloration, high gluten strength, and/or reduced bitterness. In further embodiments, the invention is directed toward identity-preserved milled grain products such as wheat flour manufactured substantially only from certain white wheat varieties, identity-preserved grain intermediate products such as milled bran flours manufactured substantially only from certain white wheat varieties, and to methods of preparation of each. In certain embodiments, such milled identity-preserved grain products require less sugar, less gluten, and/or reduced refining and bleaching during their use, thereby decreasing the cost of food production and providing certain advantageous characteristics.

### CROSS-REFERENCE TO RELATED APPLICATION

This application is a utility conversion of United States Provisional Patent Application Ser. No. 61/264,599, filed Nov. 25, 2009, for “White Wheat Varieties, and Compositions and Methods of Using the Same,” U.S. Provisional Patent Application Ser. No. 61/405,071, filed Oct. 20, 2010, for “White Wheat Varieties, and Compositions and Methods of Using the Same,” U.S. Provisional Patent Application Ser. No. 61/405,124, filed Oct. 20, 2010, for “Gluten Quality Wheat Varieties and Methods of Use.” and U.S. Provisional Patent Application Ser. No. 61/369,566, filed Jul. 30, 2010, for “Gluten Quality Wheat Varieties and Methods of Use.”

### FIELD OF THE DISCLOSURE

This invention relates generally to agriculture, and more particularly to cultivated wheat varieties and uses thereof. More specifically, the invention relates to wheat varieties that exhibit advantageous traits of white coloration, high gluten strength, and/or reduced bitterness. In further embodiments, the invention is directed toward milled grain products such as wheat flour manufactured from wheat varieties of the invention, and grain intermediate products such as milled bran flours manufactured from wheat varieties of the disclosure.

### BACKGROUND

Wheat is an important crop as a food staple and nutritional agent, and has been domesticated for about 10,000 years. In 2007, world production of wheat was 607 million tons, which makes wheat the third most-produced cereal after maize and rice. Wheat grain is a staple food used to make flour for leavened, flat, and steamed breads, biscuits, cookies, cakes, breakfast cereal, pasta, noodles, couscous, and for fermentation to make beer, alcohol, vodka, or biofuels. Wheat is also planted to a limited extent as a forage crop for livestock and as a construction material for roofing thatch.

Most white flour is milled from a mixture of different wheats. The proportion of each kind will typically depend upon a variety of factors, such as the amount and proportion of protein contained therein. During the milling process, the endosperm portion of the wheat kernels is separated from the bran layers through a series of breaking and screening steps. While the resulting bran is commonly relegated to breakfast cereals or animal feeds, the endosperm fraction is ground to separate flour from the coarser endosperm particles. Finally, the flour is treated with bleaching and aging agents, enriched with vitamins, and packaged for both domestic and commercial end-users.

In the United States, wheat is classified according to whether it is hard or soft, white or red, and winter or spring. In order to fulfill their demands, flour millers must choose between available varieties of wheats that are grown in different regions, depending upon soil and climate characteristics, and which provide different characteristic properties. For example, soft red winter wheats are typically grown in Ohio, Indiana, and areas of the Southeastern U.S. Meanwhile, soft white wheats are generally grown in the Pacific Northwest and Michigan. Hard red winter wheats are primarily grown in Kansas, Nebraska, Oklahoma, and Texas. Hard wheats typically have higher gluten strength properties that are better suited for bread baking than soft wheats. Therefore, commercial bread bakers are generally biased in favor of flours made primarily from hard wheat varieties, and these varieties are demanded by millers accordingly.

Currently, red wheat is more readily available in the United States than white wheat. Production of hard white wheat in the United States was on less than 2 million acres in 2006. Hard red wheats are characterized by a relatively strong wheat flavor that consumers may not want for whole wheat bread products. Red wheat also has a distinctive bitter taste due to the tannins and phenolic compounds in the bran that many consumers find unpleasant, and which is offset in the final baking product by the presence of expensive sweeteners. Moreover, red wheats will have a red color in the intact wheat kernel and its outer layers. The distinct red hue of whole wheat flour milled from hard red wheat varieties may be problematic for bread products like whole wheat croissants and Danish rolls that consumers typically associate with a white hue. Furthermore, bran separated from hard red wheat varieties is generally only suitable for animal feeds, and therefore is less valuable to the miller than brans derived from white wheat varieties that may be used in breakfast cereals and other bran products consumed by humans. Red wheat also may have lower milling performance compared to white wheat, because a significantly higher extraction rate may be used with white wheat without sacrificing flour color.

In conventional flour milling, the grain is subjected to a series of milling steps that each involve a break system comprised of a pair of break rolls and an associated set of sieves. Coarser fractions that are removed by the sieves are then subsequently milled by the following break system to progressively size-reduce the endosperm to produce flour. Traditional bulk systems of moving grain have been designed to facilitate economies of scale; they bring together small loads of grain into one large load. As such, traditional systems comingle different varieties of wheat grown in the field. Comingling of wheat varieties most often occurs in storage from the farm to the elevator, in storage to rail or barge, from the rail or barge to the elevator, or from the elevator to shipment.

While white wheat varieties theoretically could fill niche markets by being used to produce a white whole wheat bread with a milder flavor and more desirable coloration, white wheats are generally soft, with lower gluten strength, and are correspondingly difficult to sell to millers for baking flour. It is an aim of the present invention to provide improved white wheat varieties, and identity-preserved grain products comprising substantially only improved white wheat varieties with high gluten strength and fewer bitter tannins and phenolic compounds.

### SUMMARY OF THE INVENTION

The following embodiments are described in conjunction with systems, tools and methods which are meant to be exemplary and illustrative, and not limiting in scope. In various embodiments, white wheat varieties with favorable color characteristics (i.e., whiteness); reduced tannin levels; reduced levels of phenolic compounds; and/or high gluten strength are provided, while other embodiments are directed to other improvements. In some embodiments, “identity-preserved” milled grain products comprising a white wheat variety of the invention are provided. In certain embodiments, the white wheat varieties with favorable color characteristics (i.e., whiteness); are selected from the list consisting of AZBR81207WW, AUBR31117W, AUBR6122W, CABR5437W, AZCABR4421W, ARGIMI7232W, COI565W, AUBR31064W, AUBR31282W, CHBR1481W, and ARGBR5945W. In further embodiments, “identity-preserved” grain intermediate products comprising a white wheat variety of the invention are provided. Also provided are methods of preparing “identity-preserved” milled grain products and “identity-preserved” grain intermediate products of the invention.

Another embodiment includes a method for producing an F1 wheat seed comprising crossing a wheat plant of the invention with a different wheat plant and harvesting the resulting F1 wheat seed. A particular embodiment includes a method of producing a male sterile wheat plant comprising transforming the wheat plant with a nucleic acid molecule that confers male sterility. Yet another embodiment includes a method of producing an herbicide resistant wheat plant comprising transforming the wheat plant with a transgene that confers herbicide resistance. Embodiments of the invention include an herbicide resistant wheat plant produced by the method. A particular embodiment includes a method of producing an insect resistant wheat plant comprising transforming the wheat plant with a transgene that confers insect resistance. The invention includes an insect resistant wheat plant produced by the method.

### DETAILED DESCRIPTION

### I. Terms

In order to facilitate discussion of the various embodiments of the invention, the following explanations of specific terms are provided:

Grist: As used herein, the term “grist” refers to grain that has been separated from its chaff in preparation for grinding. It can also mean grain that has been ground at a grist mill. Grist can be ground into meal or flour, depending on how coarsely it is ground.

White wheat: As used herein, the term “white wheat” refers to wheat cultivars sufficiently white to allow discrimination from red wheats and meet grain classification standards. Whiteness may be measured either subjectively or objectively. A subjective minimum grain color standard for hard white wheat was established by the Federal Grain Inspection Service of USDA-GIPSA in 1990, when hard white wheat was officially recognized as a unique market class in the United States. The color standard was based on a grain sample for the hard white wheat variety “Klasic,” produced in California. However, this color standard was waived in 1994 when numerous samples of “Klasic” were found with grain darker than the officially accepted standard. From 1994 to 1999, an interim classification procedure was used based on variety identity and production origin. The degree of “whiteness” of a given wheat may be empirically determined using near-infrared spectroscopy (NIRS), using the visible-near-infrared wavelength range (570-1098 nm). This wavelength range is the same used by protein-testing NIRS instruments at grain receiving and shipping points. The resulting “Minolta L*value” provide a measurement of whiteness; the higher the Minolta L*value, the greater is the degree of whiteness. Klasic standard white wheat was found to have an L*value of 41.35. Peterson et al. (2001)/Euphytica 119:101-6.

Identity-preserved: As used herein, the term “identity-preserved” refers to grain or grain products wherein the identity of the grain, or grain from which the grain product was produced, is preserved from field to customer. The identity preservation of grains involves a system of production and delivery in which the grain is segregated based on intrinsic characteristics (such as variety or production process) during all stages of production, storage, and transportation. The development of an identity-preserved grain or grain product allows for the grain or grain product to be marketed by reference to its specific attributes, rather than merely by its classification. Thus, identity-preserved grain or grain products can satisfy niche markets according to specific consumer demands for, inter alia, organic, genetically-modified, whiteness, high gluten strength, reduced sugar, and/or unrefined grain or grain products.

Grain product: As used herein, the term “grain product” refers to compositions comprising one or more constituents of one or more grains. Grain constituents include any component of a whole grain, e.g., the whole grain kernel, the germ, the bran, the endosperm, and any combination thereof. Whole grains typically refer to the germ, bran, and endosperm of a grain, and may be milled or unmilled. Refined grains typically refer to grain products in which the bran and most of or the entire germ have been removed, leaving primarily or only the endosperm. A grain product may be, for example, any combination of one or more components of a grain that have been ground into flour, cut into pieces of a variety of sizes, or used whole.

Milled grain product: Wheat milling is a mechanical method of breaking open the wheat kernel to separate as much endosperm as possible from the bran and germ, and to grind the endosperm into flour. This process substantially separates the major components of wheat from one another. As used herein, the term “milled grain product” refers to compositions comprising endosperm separated from other major components of wheat by the milling process. Refined wheat flour is produced when most of the bran and germ are separated from the endosperm.

Grain intermediate product: As used herein, the term “grain intermediate product” refers to compositions comprising wheat components other than endosperm that has been separated from the endosperm by the milling process. Bran and germ are non-limiting examples of grain intermediate products.

### II. White Wheat Varieties with Desired Characteristics

White wheat is the newest class of wheat to be marketed in the United States. Hard white wheat was added as a U.S. market class in 1990. White wheat contains the same healthy levels of while grain fiber that red wheat does, but does not have as strong a flavor or dark color. White wheat may be actually golden in color. It tastes sweeter and is lighter than its hard red wheat counterparts. White wheat is planted like red wheat, grows like red wheat, and produces similar yields to red wheat. The difference between red and white wheat is the color of the seed coat. The differences between hard and soft white wheat are found mainly in the end products for which they are used; soft white has a lower protein level than hard white. Thus, soft white wheat is used mainly for bakery products other than bread. Examples include pastries, cakes, and cookies. Soft white wheat is also used for cereals, flat breads, and crackers.

Hard white wheat can be used for the same products as hard red wheat. Hard white wheat is used, for example, in whole-wheat and high-extraction flour applications. Bakers like it because hard white wheats are excellent for use in the bread making industry. Because it has a naturally sweeter flavor, bakers can use less sweeteners. International customers prefer it for at least two reasons: 1) higher extraction of white wheat flour while maintaining its bright white color; and 2) most white wheat gives better color stability in Asian wet noodles. Hard white wheat is a superior ingredient for all yeast breads, Artisan breads, Asian noodles, tortillas, pizza crusts, breadsticks, flatbreads, quick breads, and more.

Both white wheat classes make quality 100% whole wheat products. Whole-wheat products made from white wheat have a favorable appearance, when compared with similar products made from red wheat, since they have less pigmentation. Additionally, with fewer phenolic compounds and tannins in the bran, white wheat imparts a less bitter taste to the final product. Whole-wheat breads made with white wheat have a similar taste and appearance to bread made from refined red wheat flour. Therefore, substitution of white wheat for red wheat allows refining and bleaching of the flour to be reduced or eliminated, while still meeting consumers' expectations about the finished product's characteristics.

In some embodiments of the invention, white wheat varieties are provided with superior characteristics compared to prior art white wheat varieties. The white wheat varieties of these embodiments may be preferred by commercial and domestic bakers and users of wheat products. In some embodiments, the provided white wheat varieties exhibit some or all of the following improvements over prior available varieties of white wheat: higher degree of whiteness; higher gluten strength; lower levels of phenolic compounds; lower levels of tannins; lower requirement for added sugar in flour made from the provided white wheat varieties; and lower requirement for bleaching or refinement of flour made from the provided white wheat varieties.

Grain products produced from white wheat varieties of the invention may exhibit one or more advantageous properties. In some embodiments, a grain product produced from a white wheat variety with high gluten strength requires less added gluten before dough making Consequently, the cost of using the grain product of these embodiments is decreased, due to the saved expense of purchasing additional gluten. In some embodiments, a grain product produced from a white wheat variety having fewer phenolic compounds is less bitter, and therefore requires less added sweetener before use. The cost of using the grain product of these embodiments is decreased, due to the saved expense of purchasing additional sweetener. In some embodiments, a grain product produced from a white wheat variety with fewer tannins is less bitter, and therefore requires less added sweetener before use. The cost of using the grain product of these embodiments is decreased, due to the saved expense of purchasing additional sweetener.

Management practices that optimize the production of the white wheat varieties provided herein are similar to those used for hard red wheat. Although end users require a range in protein content for their products made from white wheat, in most cases high-protein white wheat is preferred. Therefore, nitrogen management and variety choice should focus on producing a crop with high grain protein. Fungicide applications for foliar diseases and/or scab may be required in disease-prone regions and in seasons with high disease pressure.

White wheat varieties are generally more prone to preharvest sprouting than their red wheat counterparts. Rain, high humidity, and cool temperatures after the grain has matured can induce sprouting in the spike. Hard white wheat should be harvested in a timely manner to reduce preharvest sprouting. Harvesting white wheat before hard red spring wheat varieties is one way of reducing the chance of unnecessary exposure to conditions that may induce preharvest sprouting.

### III. Identity-Preserved Grain Products

According to particular embodiments of the invention, there are provided identity-preserved milled grain products. In one such embodiment, a milled grain product is produced wherein the white wheat grain has been segregated from red wheat and other varieties of white wheat during all stages of storage, transportation, and production, e.g., milling.

Millers typically blend different wheats to achieve the desired grain end product. In some embodiments, a white wheat variety of the invention is segregated from other varieties during milling. Thus, in each of the milling steps of inspection and storage, cleaning, and conditioning, a white wheat variety of the invention is kept separate from other wheat varieties that would otherwise contaminate the process.

Inspection and Storage

Wheat typically arrives at a mill by truck, ship, barge, or rail car. Before the wheat is unloaded, samples are taken to be sure it passes inspection. X-rays may be used to detect any signs of insect infestation. Meanwhile, product control chemists may begin tests to classify the grain by milling and baking a small amount to determine end-use qualities. The results from these tests determine how the wheat will be handled and stored. The wheat will then be stored at the mill in large bins. Storing wheat is an exact science practiced by skilled artisans. The right moisture, heat, and air must be maintained, or the wheat may mildew, sprout, or ferment.

Cleaning the Wheat

The first milling steps involve cleaning the wheat; equipment separates wheat from seeds and other grains, eliminates foreign materials such as metal, sticks, stones, and straw, and scours each kernel of wheat. Cleaning can take as many as, for example, six steps: (1) Magnetic Separator—the wheat first passes by a magnet that removes iron and steel particles; (2) Separator—vibrating screens remove bits of wood and straw and almost anything too big or too small to be wheat; (3) Aspirator—air currents act as a kind of vacuum to remove dust and lighter impurities; (4) De-stoner—using gravity, a machine separates the heavy material from the light material to remove stones that may be the same size as wheat kernels; (5) Disc separator—the wheat passes through a separator that identifies the size of the kernels even more closely, rejecting anything longer, shorter, more round, more angular, or in any way shaped differently than an expected kernel; and (6) Scourer—the scourer removes outer husks, crease dirt, and any smaller impurities with an intense scouring action, while currents of air pull substantially all the loosened material away.

Conditioning the Wheat

The wheat is conditioned for milling through a process called “tempering.” Moisture is added in precise amounts to toughen the bran and mellow the inner endosperm. This makes the parts of the kernel separate more easily and cleanly. Tempered wheat is stored in bins from 8 to 24 hours, depending on the type of wheat (soft, medium, or hard). Blending of wheats typically is done at this time to achieve the best flour for a specific end-use.

In an impact scourer/entoleter, centrifugal force then breaks apart any unsound kernels and rejects them from the mill flow. From the entoleter, the wheat flows to grinding bins-large hoppers that will measure or feed wheat to the actual milling process.

After passing through the entoleter, the wheat kernels, or berries, are in better condition than when they arrived at the mill and are ready to be milled into flour. Wheat kernels are measured or fed from the bins to the “rolls,” or corrugated rollers made from chilled cast iron. The rolls are paired and rotate inward against each other, moving at different speeds. Just one pass through the corrugated “first break” rolls begins the separation of bran, endosperm and germ. This modern milling process is a gradual reduction of wheat kernels. The goal is to produce middlings, or coarse particles of endosperm. The middlings are then graded and separated from the bran by sieves and purifiers. Each size returns to the corresponding rollers and the same process is repeated until the desired flour is obtained.

The miller's skill is demonstrated by the ability to adjust all of the rolls to the proper settings that will produce the maximum amount of high-quality flour. Grinding too hard or close results in bran powder in the flour. Grinding too open allows good endosperm to be lost in the mill's feed system. The miller must select the exact milling surface, or corrugation, on the break rolls, as well as the relation and the speed of the rollers to each other to match the type of wheat and its condition. Each break roll must be set to get as much pure endosperm as possible to the middlings rolls. The middlings rolls are set to produce as much flour as possible.

From the rolls, the grist is sent upwards to drop through sifters. The grist is moved via pneumatic systems that mix air with the particles so they flow through tubes. This is a superior method in terms of health and safety over earlier methods of moving the grist with buckets.

The broken particles of wheat are introduced into rotating sifters where they are shaken through a series of bolting cloths or screens to separate the larger from the smaller particles. Inside the sifter, there may be as many as, for example, 27 frames, each covered with either a nylon or stainless steel screen, with openings that get smaller the farther they go down. Up to, for example, about six different sizes of particles may come from a single sifter, including some flour with each sifting. Larger particles are shaken off from the top, or “scalped,” leaving the finer flour to sift to the bottom. The scalped fractions are sent to other roll passages and particles of endosperm are graded by size and carried to separate purifiers.

In a purifier, a controlled flow of air lifts off bran particles while at the same time a bolting cloth separates and grades coarser fractions by size and quality.

About four or five additional break rolls, each with successively finer corrugations and each followed by a sifter, are usually used to rework the coarse stocks from the sifters and reduce the wheat particles to granular middlings that are as free from bran as possible. Germ particles will be flattened by later passage through the smooth reduction rolls and can easily be separated. The reduction rolls reduce the purified, granular middlings, or farina, to flour. The process is repeated, sifters to purifiers to reducing rolls, until the maximum amount of flour is separated, consisting of, for example, about 75% of the wheat.

There are various grades of flour produced in the milling process. Bakers buy a wide variety of flour types, based on the products they produce. The flour the consumer buys at the grocery store, called “family flour” by the milling industry, is usually a long-patent all-purpose or bread flour. Occasionally, short patent flour is available in retail stores. “Reconstituting,” or blending back together, all the parts of the wheat in the proper proportions yields whole wheat flour. This process produces higher quality whole wheat flour than is achieved by grinding the whole wheat berry. Reconstitution assures that the wheat germ oil is not spread throughout the flour so it does not readily go rancid.

The remaining percentage of the wheat kernel or berry is classified as millfeed-shorts, bran, and germ. These are examples of grain intermediate products. In some embodiments, improved white wheat varieties of the invention are kept segregated from other wheat varieties during milling and all handling of the wheat prior to milling. In these embodiments, grain intermediate products obtained from the milling of that identity-preserved wheat are kept segregated from any grain intermediate products produced by milling other varieties of wheat, thereby yielding identity-preserved grain intermediate products.

Toward the end of the line in the millstream, if the flour is to be “bleached,” the finished flour flows through a device, which releases a bleaching-maturing agent in measured amounts. In the bleaching process, flour is exposed to chlorine gas or benzoyl peroxide to whiten and brighten the flour color. In some embodiments of the invention, flour produced from an improved variety of white wheat does not require bleaching, because the flour has a natural white color. This represents a desired result, as consumers may prefer an unbleached flour with the same pleasing color characteristics as standard bleached wheat flour.

The flour stream next passes through a device that measures out specified amounts of enrichment. The enrichment of flour with four B vitamins (thiamin, niacin, riboflavin) and iron began in the 1930s. In 1998, folate, or folic acid, was added to the mix of vitamin B. If the flour is self-rising, a leavening agent, salt, and calcium are also added in specified amounts.

Before the flour leaves the mill, additional lab tests are generally run to ensure that the flour conforms to the purchaser's specifications. Finally, the millstream typically flows through pneumatic tubes to the packing room or into hoppers for bulk storage. Family flour for retail sale may be packaged in, for example, from about 5 to about 25 pound bags. Bakery flour may be packaged in, for example, from about 50 to about 100 pound bags, or sent directly to bulk trucks or rail cars.

Likewise, by means of the present invention, agronomic genes can be expressed in plants of the present invention. More particularly, plants can be genetically engineered to express various phenotypes of agronomic interest. Exemplary genes implicated in this regard include, but are not limited to, those categorized below:

### 1. Genes That Confer Resistance to Pests or Disease and That Encode:

A. Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance genes to engineer plants that are resistant to specific pathogen strains. See, for example, Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A gene conferring resistance to a pest, such as soybean cyst nematode. See e.g., PCT Application WO 96/30517; PCT Application WO 93/19181.

C. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

D. A lectin. See, for example, the disclosure by Van Damme et al., Plant Molec. Biol. 24:25 (1994), who disclose the nucleotide sequences of several Clivia miniata mannose-binding lectin genes.

E. A vitamin-binding protein such as avidin. See PCT application US93/06487. The application teaches the use of avidin and avidin homologues as larvicides against insect pests.

F. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor); Huub et al., Plant Molec. Biol. 21:985 (1993) (nucleotide sequence of cDNA encoding tobacco proteinase inhibitor I); Sumitani et al., Biosci. Biotech. Biochem. 57:1243 (1993) (nucleotide sequence of Streptomyces nitrosporeus .alpha.-amylase inhibitor); and U.S. Pat. No. 5,494,813 (Hepher and Atkinson, issued Feb. 27, 1996).

G. An insect-specific hormone or pheromone such as an ecdysteroid or juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor); and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

I. An insect-specific venom produced in nature by a snake, a wasp, etc. For example, see Pang et al., Gene 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase; and Kawalleck et al., Plant Molec. Biol. 21:673 (1993), who provide the nucleotide sequence of the parsley ubi4-2 polyubiquitin gene.

L. A molecule that stimulates signal transduction. For example, see the disclosure by Botella et al., Plant Molec. Biol. 24:757 (1994), of nucleotide sequences for mung bean calmodulin cDNA clones; and Griess et al., Plant Physiol. 104:1467 (1994), who provide the nucleotide sequence of a maize calmodulin cDNA clone.

M. A hydrophobic moment peptide. See PCT application WO 95/16776 (disclosure of peptide derivatives of Tachyplesin which inhibit fungal plant pathogens) and PCT application WO 95/18855 (teaches synthetic antimicrobial peptides that confer disease resistance).

N. A membrane permease, a channel former or a channel blocker. For example, see the disclosure of Jaynes et al., Plant Sci. 89:43 (1993), of heterologous expression of a cecropin-β, lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

P. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

Q. A virus-specific antibody. See, for example, Tavladoraki et al., Nature 366:469 (1993), who show that transgenic plants expressing recombinant antibody genes are protected from virus attack.

R. A developmental-arrestive protein produced in nature by a pathogen or a parasite. Thus, fungal endo a-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

S. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

### 2. Genes That Confer Resistance to an Herbicide:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988); and Mild et al., Theon. Appl. Genet. 80:449 (1990), respectively.

B. Glyphosate (resistance conferred by, e.g., mutant 5-enolpyruvylshikimate-3-phosphate synthase (EPSPs) genes (via the introduction of recombinant nucleic acids and/or various forms of in vivo mutagenesis of native EPSPs genes), aroA genes and glyphosate acetyl transferase (GAT) genes, respectively), other phosphono compounds such as glufosinate (phosphinothricin acetyl transferase (PAT) genes from Streptomyces species, including Streptomyces hygroscopicus and Streptomyces viridichromogenes), and pyridinoxy or phenoxy proprionic acids and cyclohexones (ACCase inhibitor-encoding genes), See, for example, U.S. Pat. No. 4,940,835 to Shah, et al. and U.S. Pat. No. 6,248,876 to Barry et. al., which disclose nucleotide sequences of forms of EPSPs which can confer glyphosate resistance to a plant. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al., DeGreef et al., Bio/Technology 7:61 (1989), describe the production of transgenic plants that express chimeric bar genes coding for PAT activity. Exemplary of genes conferring resistance to phenoxy proprionic acids and cyclohexones, such as sethoxydim and haloxyfop are the Accl-S1, Accl-S2 and Accl-S3 genes described by Marshall et al., Theor. Appl. Genet. 83:435 (1992). GAT genes capable of conferring glyphosate resistance are described in WO 2005012515 to Castle et. al. Genes conferring resistance to 2,4-D, fop and pyridyloxy auxin herbicides are described in WO 2005107437 and U.S. patent application Ser. No. 11/587,893, both assigned to Dow AgroSciences LLC.

C. An herbicide that inhibits photosynthesis, such as a triazine (psbA and gs+ genes) or a benzonitrile (nitrilase gene). Przibila et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

### 3. Genes That Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al., Proc. Natl. Acad. Sci. U.S.A. 89:2624 (1992).

B. Decreased phytate content-1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene. 2) A gene could be introduced that reduced phytate content. In maize for example, this could be accomplished by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteol. 170:810 (1988) (nucleotide sequence of Streptococcus mutants fructosyltransferase gene); Steinmetz et al., Mol. Gen. Genet. 20:220 (1985) (nucleotide sequence of Bacillus subtilis levansucrase gene); Pen et al., Bio/Technology 10:292 (1992) (production of transgenic plants that express Bacillus licheniformis α-amylase); Elliot et al., Plant Molec. Biol. 21:515 (1993) (nucleotide sequences of tomato invertase genes); Sogaard et al., J. Biol. Chem. 268:22480 (1993) (site-directed mutagenesis of barley α-amylase gene); and Fisher et al., Plant Physiol. 102:1045 (1993) (maize endosperm starch branching enzyme II).

D. Abiotic Stress Tolerance which includes resistance to non-biological sources of stress conferred by traits such as nitrogen utilization efficiency, altered nitrogen responsiveness, drought resistance cold, and salt resistance. Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress.

The examples presented herein are provided for illustrative purposes only and not to limit the scope of any embodiment of the present invention.

### EXAMPLES

Eleven white wheat breeding lines (i.e., AZBR81207WW, AUBR31117W, AUBR6122W, CABR5437W, AZCABR4421W, ARGIMI7232W, COI565W, AUBR31064W, AUBR31282W, CHBR1481W, and ARGBR5945W) and one commercially registered variety of white wheat (Kansas Diamond) were milled to produce whole grain (WG) flour. The flour was then analysed for color through a variety of tests. Baking characteristics were evaluated using a micro test baking method. The samples were designated by the following Batch numbers:

WHITE WHEAT SAMPLES

| BATCH # | VARIETY |
|---|---|
| A | AZBR81207WW |
| B | AUBR31117W |
| C | AUBR6122W |
| D | CABR5437W |
| E | AZCABR4421W |
| F | KANSAS DIAMOND |
| G | ARGIMI7232W |
| H | COI565W |
| I | AUBR31064W |
| J | AUBR31282W |
| K | CHBR1481W |
| L | ARGBR5945W |

### Example 1

### Milling

Samples were tempered to 15.5% moisture content for 20 hrs and then milled on a Brabender Quadramat Jr. according to internal procedures established at CIGI. The bran fraction was then reduced using a pin mill (20,000 rpm) and added back to the milled flour to produce WG flour. The granulation of the WG flour was evaluated using an appropriate series of standard sieves on a Baler laboratory sifter strength, content, or quality.

### Example 2

### Sample Analysis

Wheat samples were analyzed for particle size index (PSI; AACC 55-30) to determine kernel hardness characteristics. Flour samples were analyzed for protein content (Williams et al., 1998), farinograph (AACC 54-21, constant flour weight procedure, 50 g bowl), moisture content (AACC 44-15A, one-stage procedure) and color using a dry packing method (Minolta; internal procedures; D65 wavelength).

### Example 3

### Baking Performance

Micro test baking performance of WG flour samples were evaluated based on the no time dough (NTD) process. Flour samples (35 g) were treated with sugar (8%), salt (2%), canola oil (3%), yeast (4%), ammonium phosphate (0.1%), ascorbic acid (60 ppm) and amylase (60 ppm). Flour was mixed to peak dough development time plus an additional 10% as determined using RAR software for capturing mixer energy input. The optimally mixed dough was rested for 10 min, scaled at 40 g, rounded, rested an additional 10 min, sheeted thru a 3/16 and a ⅛ inch gap and finally molded before being placed in the proofer (37° C./98.6° F., 85% RH). Proofing time was set as the amount of time a dummy WG dough (CWRS) took to reach 48 mm in height plus an additional 2 min due to the inherently stronger dough properties of the CWRS class of wheat. Fully proofed doughs were baked for 20 min at 375° F. Samples were cooled, and then evaluated for loaf volume by TexVol, external appearance, internal crumb color and internal texture according to established CIGI procedures.

### Example 4

### Results

All samples had similar granulation, with the exception of sample G which showed a slightly higher amount of product over 340 μm and a lower amount of product with granulation over 212 μm.

Analytical results are presented in Tables 1a through 1c.

TABLE 1a
Milling and Analytical Results (samples A through D)

| | Sample | | | |
|---|---|---|---|---|
| | A | B | C | D |
| ID No. | W402-10 | W403-10 | W404-10 | W405-1 |

WHEAT (13.5% mb)

| | | | | |
|---|---|---|---|---|
| Moisture, % | 10.1 | 10.4 | 10.8 | 9.3 |
| Particle size | 37 | 47 | 46 | 40 |
| index, % | | | | |

MILLING YIELD

| | | | | |
|---|---|---|---|---|
| Flour yield | 100.0 | 100.0 | 100.0 | 100.0 |
| (Whole | | | | |
| grain flour), % | | | | |

GRANULATION

| | | | | |
|---|---|---|---|---|
| Over 60w (340 μm), % | 11 | 11 | 12 | 11 |
| Over 6xx (212 μm), % | 13 | 10 | 11 | 13 |
| Over 9xx (150 μm), % | 23 | 24 | 25 | 25 |
| Thrus, % | 53 | 55 | 52 | 51 |
| Total | 100 | 100 | 100 | 100 |
| recovery, % | | | | |

FLOUR (14.0% mb)

| | | | | |
|---|---|---|---|---|
| Protein | 15.2 | 13.3 | 13.0 | 13.0 |
| content | | | | |
| (CNA), % | | | | |
| Wet gluten, % | 37.9 | 33.0 | 33.7 | 32.0 |
| Minolta | 82.8 | 84.8 | 85.3 | 84.7 |
| color - L* | | | | |
| a* | 1.49 | 0.78 | 0.65 | 0.86 |
| b* | 12.7 | 10.5 | 11.2 | 10.9 |
| Moisture, % | 12.7 | 12.9 | 12.8 | 13.2 |

TABLE 1b
Milling and Analytical Results (samples E through H)

| Sample | | | |
|---|---|---|---|
| E | F | G | H |

WHEAT (13.5% mb)

| | | | | |
|---|---|---|---|---|
| Moisture, % | 11.3 | 10.2 | 9.8 | 9.2 |
| Particle size | 47 | 44 | 64 | 48 |
| index, % | | | | |

MILLING YIELD

| | | | | |
|---|---|---|---|---|
| Flour yield | 100.0 | 100.0 | 100.0 | 100.0 |
| (Whole | | | | |
| grain flour), % | | | | |

GRANULATION

| | | | | |
|---|---|---|---|---|
| Over 60w (340 μm), % | 12 | 11 | 16 | 12 |
| Over 6xx (212 μm), % | 10 | 12 | 5 | 12 |
| Over 9xx (150 μm), % | 24 | 25 | 23 | 22 |
| Thrus, % | 54 | 52 | 56 | 54 |
| Total | 100 | 100 | 100 | 100 |
| recovery, % | | | | |

FLOUR (14.0% mb)

| | | | | |
|---|---|---|---|---|
| Protein | 13.4 | 11.1 | 11.3 | 13.5 |
| content | | | | |
| (CNA), % | | | | |
| Wet gluten, % | 33.1 | 29.0 | 32.9 | 34.6 |
| Minolta | 85.1 | 85.4 | 88.1 | 85.5 |
| color - L* | | | | |
| a* | 0.70 | 0.78 | 0.42 | 0.75 |
| b* | 11.5 | 10.4 | 7.3 | 10.3 |
| Moisture, % | 12.4 | 12.5 | 12.0 | 12.7 |

TABLE 1c
Milling and Analytical Results (samples I through L)

| Sample | | | |
|---|---|---|---|
| I | J | K | L |

WHEAT (13.5% mb)

| | | | | |
|---|---|---|---|---|
| Moisture, % | 11.0 | 10.5 | 10.2 | 10.0 |
| Particle size | 41 | 49 | 40 | 45 |
| index, % | | | | |

MILLING YIELD

| | | | | |
|---|---|---|---|---|
| Flour yield | 100.0 | 100.0 | 100.0 | 100.0 |
| (Whole | | | | |
| grain flour), % | | | | |

GRANULATION

| | | | | |
|---|---|---|---|---|
| Over 60w (340 μm), % | 11 | 12 | 11 | 9 |
| Over 6xx (212 μm), % | 12 | 12 | 14 | 14 |
| Over 9xx (150 μm), % | 23 | 20 | 23 | 23 |
| Thrus, % | 54 | 56 | 52 | 54 |
| Total | 100 | 100 | 100 | 100 |
| recovery, % | | | | |

FLOUR (14.0% mb)

| | | | | |
|---|---|---|---|---|
| Protein | 13.2 | 17.1 | 13.7 | 12.6 |
| content | | | | |
| (CNA), % | | | | |
| Wet gluten, % | 34.3 | 49.2 | 33.9 | NES |
| Minolta | 85.1 | 85.6 | 84.7 | 84.1 |
| color - L* | | | | |
| a* | 0.88 | 0.72 | 0.91 | 0.75 |
| b* | 10.8 | 9.9 | 10.5 | 11.0 |
| Moisture, % | 12.2 | 11.9 | 12.5 | 12.6 |

Similar kernel hardness, as evidenced by PSI results, was observed for all samples with the exception of sample G, which was observed to have kernel hardness characteristics similar to soft wheat, as shown in Tables 1a through 1c.

A wide range of protein content was observed among the samples. The lowest protein content was observed for samples F and G (approximately 11%), while the highest protein content was observed for sample J (17.1%). The majority of the samples tended to have protein contents around 13%.

Color was assessed using the Minolta colorimeter which measures darkness (0) to brightness (100) in terms of L*, redness (+values) to greenness (− values) in terms of a* and yellowness (+values) to blueness (− values) in terms of b* values. From past experience, a 1 unit difference in L* values is noticeable to the human eye. Sample G was observed to have the highest level of brightness (L*) and lowest level of yellowness (b*). The lowest level of brightness (L*) was observed for sample A. This sample also had the highest level of redness (a*) and yellowness (b*) values. Examination of the kernels of this sample showed characteristics similar to that of amber durum wheat.

The 35 g micro test baking method provided useful data and exposed clear differences among the samples for volume, functionality, and whiteness (Tables 3a-3c). Mixing times of the samples ranged from 4.0-10.1 min, and were best explained by GI results as opposed to protein content (Tables 3a to 3c). This supports the use of GI as a useful indicator of dough strength, and supports the role of protein content for explaining baking absorption requirements. All samples processed well, but some of the samples, specifically A, D, E, I and J, showed stronger dough properties and improved handling. Dough handling indicated ease of processing the flour samples, but did not directly translate into improved loaf volume. For example, samples H and L exhibited weaker dough handling, average loaf volume (LV) and long mixing times, however, samples A and D exhibited strong dough handling, but below average LV and shorter mixing times. LV results ranged from 103 cc for sample A to 122 cc for sample J, however no apparent relationship between LV and other quality parameters was observed. The whitest crumb color was seen in samples G, J and K, while the darkest crumb color was seen in samples C, A, and L.

TABLE 3a
Test Baking Results (samples A through D)

| Sample | | | |
|---|---|---|---|
| A | B | C | D |

TEST BAKING (NO TIME DOUGH)

| | | | | |
|---|---|---|---|---|
| Baking | 72.9 | 68.5 | 68.3 | 73.7 |
| absorption, % | | | | |
| Mixing time, | 6.37 | 8.9 | 8.5 | 7.9 |
| min | | | | |
| Power, watt | 13.7 | 17.3 | 15.4 | 16.2 |
| Handling | Strong | Slight Weak | Weak | Strong |
| Comments | | | | |
| Loaf volume | 103 | 116 | 105 | 104 |
| (TexVol), cc | | | | |
| Specific | 2.6 | 3.0 | 2.7 | 2.7 |
| volume, | | | | |
| (TexVol), | | | | |
| cc/g bread | | | | |
| External | 20 | 15 | 16 | 18 |
| score (out of | | | | |
| 30) | | | | |
| Internal | 43 | 42 | 42 | 41 |
| score(out of | | | | |
| 60) | | | | |
| Total Bread | 63 | 57 | 58 | 59 |
| Score (out | | | | |
| of 90) | | | | |
| L*, | 66.6 | 66.2 | 65.9 | 65.8 |
| (Minolta) | | | | |
| Whiteness | 11 | 8 | 12 | 9 |
| order, visual | | | | |
| score | | | | |
| Cell | 0.66 | 0.71 | 0.66 | 0.71 |
| contrastb | | | | |
| Cell diameter_{c}, | 2.16 | 1.85 | 1.94 | 1.79 |
| mm | | | | |
| Cell wall | 0.493 | 0.462 | 0.457 | 0.460 |
| thickness_{d}, | | | | |
| mm | | | | |
| Slice area, | 1674 | 1633 | 1725 | 1521 |
| mm2 | | | | |
| # of | 1.83 | 1.54 | 1.63 | 1.59 |
| cells/areaₑ, | | | | |
| cells/mm₂ | | | | |

TABLE 3b
Test Baking Results (samples E through H)

| Sample | | | |
|---|---|---|---|
| E | F | G | H |

TEST BAKING (NO TIME DOUGH)

| | | | | |
|---|---|---|---|---|
| Baking | 69.4 | 65.5 | 63.5 | 68.5 |
| absorption, % | | | | |
| Mixing time, | 7.4 | 5.0 | 4.0 | 8.0 |
| min | | | | |
| Power, watt | 19.5 | 14.1 | 15.0 | 14.1 |
| Handling | Strong | Good | Good | Weak |
| Comments | | | | |
| Loaf volume | 114 | 112 | 104 | 110 |
| (TexVol), cc | | | | |
| Specific | 3.0 | 2.9 | 2.7 | 2.8 |
| volume, | | | | |
| (TexVol), cc/g | | | | |
| bread | | | | |
| External | 20 | 23 | 21 | 21 |
| score (out of | | | | |
| 30) | | | | |
| Internal | 43 | 45 | 45 | 45 |
| score(out of | | | | |
| 60) | | | | |
| Total Bread | 63 | 68 | 66 | 66 |
| Score (out | | | | |
| of 90) | | | | |
| L*, | 66.6 | 69.9 | 70.2 | 69.2 |
| (Minolta) | | | | |
| Whiteness | 7 | 5 | 3 | 4 |
| order, visual | | | | |
| score | | | | |
| Cell | 0.69 | 0.70 | 0.67 | 0.71 |
| contrastb | | | | |
| Cell diameter_{c}, | 2.02 | 1.80 | 2.10 | 1.78 |
| mm | | | | |
| Cell wall | 0.469 | 0.451 | 0.477 | 0.452 |
| thickness_{d}, | | | | |
| mm | | | | |
| Slice area, | 1725 | 1525 | 1837 | 1635 |
| mm2 | | | | |
| # of | 1.73 | 1.50 | 1.71 | 1.48 |
| cells/areaₑ, | | | | |
| cells/mm₂ | | | | |

TABLE 3c
Test Baking Results (samples I through L)

| Sample | | | |
|---|---|---|---|
| I | J | K | L |

TEST BAKING (NO TIME DOUGH)

| | | | | |
|---|---|---|---|---|
| Baking | 72.0 | 77.4 | 69.5 | 71.4 |
| absorption, % | | | | |
| Mixing time, | 10.1 | 5.1 | 8.1 | 6.8 |
| min | | | | |
| Power, watt | 17.5 | 23.3 | 16.3 | 13.9 |
| Handling | Strong | Strong | Good | Weak |
| Comments | | | | |
| Loaf volume | 106 | 122 | 117 | 111 |
| (TexVol), cc | | | | |
| Specific | 2.7 | 3.2 | 3.0 | 2.9 |
| volume, | | | | |
| (TexVol), | | | | |
| cc/g bread | | | | |
| External | 19 | 21 | 23 | 17 |
| score (out of | | | | |
| 30) | | | | |
| Internal | 43 | 48 | 50 | 41 |
| score(out of | | | | |
| 60) | | | | |
| Total Bread | 62 | 69 | 73 | 58 |
| Score (out | | | | |
| of 90) | | | | |
| L*, | 67.7 | 70.3 | 71.5 | 65.5 |
| (Minolta) | | | | |
| Whiteness | 6 | 2 | 1 | 10 |
| order, visual | | | | |
| score | | | | |
| Cell | 0.69 | 0.72 | 0.68 | 0.73 |
| contrastb | | | | |
| Cell diameter_{c}, | 1.93 | 1.74 | 1.95 | 1.64 |
| mm | | | | |
| Cell wall | 0.476 | 0.452 | 0.475 | 0.441 |
| thickness_{d}, | | | | |
| mm | | | | |
| Slice area, | 1534 | 1490 | 1646 | 1715 |
| mm₂ | | | | |
| # of | 1.72 | 1.50 | 1.67 | 1.33 |
| cells/areaₑ, | | | | |
| cells/mm₂ | | | | |

The highest level of flour brightness (L*) was observed in sample G which was also observed to have a high level of crumb whiteness in the baked loaf. The remaining samples had similar flour brightness with the exception of sample A which had the lowest level of brightness. Sample A was also observed to have low crumb whiteness, ranking 11 out of 12 (12 having the lowest level of whiteness).

Deposits of the Dow AgroSciences and World Wide Wheat proprietary wheat cultivars AZCABR4421W, ARGIMI7232W, COI565W, AUBR31064W, AUBR31282W, CHBR1481W, and ARGBR5945W disclosed above and recited in the appended claims has been made with the American Type Culture Collection (ATCC), 10801 University Boulevard, Manassas, Va. 20110. The date of deposit was Nov. 11, 2010. The deposit of 2500 seeds for each variety were taken from the same deposit maintained by Dow AgroSciences since prior to the filing date of this application. All restrictions upon the deposit have been removed, and the deposit is intended to meet all of the requirements of 37 C.F.R. §1.801-1.809. The ATCC accession number for AZCABR4421W is PTA______, for ARGIMI7232W is PTA______, for COI565W is PTA______, for AUBR31064W is PTA______, for AUBR31282W is PTA______, for CHBR1481W is PTA______, and for ARGBR5945W is PTA______. The deposit will be maintained in the depository for a period of 30 years, or 5 years after the last request, or for the effective life of the patent, whichever is longer, and will be replaced as necessary during that period.

While this invention has been described in certain example embodiments, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

What is claimed is:

1. A white wheat seed designated AZCABR4421W, ARGIMI7232W, COI565W, AUBR31064W, AUBR31282W, CHBR1481W, or ARGBR5945W.

2. A wheat plant produced from the seed of claim 1.

3. A tissue culture of cells from the plant of claim 2.

4. Tissue culture as recited in claim 3, comprising regenerable cells of a plant part selected from meristematic tissue, anthers, leaves, embryos, pollen, kernel, head, stem, root, root tip, ovule, flower, and protoplasts therefrom.

5. A wheat plant regenerated from the regenerable cells of the tissue culture of claim 4.

6. Grain harvested from the plant of claim 2.

7. A grain product produced from grain harvested from the plant of claim 2.

8. The grain product of claim 7, wherein the grain product is a milled grain product.

9. The grain product of claim 7, wherein the grain product is a grain intermediate product.

10. The milled grain product of claim 8, wherein the grain product is an identity-preserved milled grain product.

11. The grain intermediate product of claim 9, wherein the grain intermediate product is an identity-preserved grain intermediate product.

12. The milled grain product of claim 8, wherein the milled grain product has one or more of the characteristics of high whiteness, high gluten strength, and low bitterness.

13. The milled grain product of claim 8, wherein the milled grain product is of substantially the same color as bleached red wheat milled grain product.

14. A method for producing an F1 wheat seed, comprising crossing the plant of claim 2, with a different wheat plant and harvesting the resulting F1 wheat seed.

15. A method of producing a male sterile wheat plant comprising transforming the wheat plant of claim 2, with a nucleic acid molecule that confers male sterility.

16. A method of producing an herbicide resistant, insect resistant, or abiotic stress tolerant wheat plant comprising transforming the wheat plant of claim 2, with a transgene that confers herbicide resistance, insect resistance or abiotic stress tolerance.

17. An herbicide resistant wheat plant produced by the method of claim 16.

18. An insect resistant wheat plant produced by the method of claim 16.

19. The wheat plant of claim 18, wherein the transgene encodes a Bacillus thuringiensis endotoxin.

20. A flour produced from grain harvested from a white wheat variety selected from the group consisting of AZCABR4421W, ARGIMI7232W, COI565W, AUBR31064W, AUBR31282W, CHBR1481W, and ARGBR5945W.

21. A food product made from the flour of claim 20.

22. The food product of claim 21 selected from the group consisting of bread, cake, or pasta.
